(19) United States
(12) Patent Application Publication
(10) Pub. No.: US 2018/0228477 A1
(43) Pub. Date: Aug. 16, 2018

(54) EXFOLIATION TOOL AND LIP BALM

(71) Applicant: Nurse Jamie, Inc., Santa Monica, CA (US)

(72) Inventor: Jamie Sherrill, Santa Monica, CA (US)

(21) Appl. No.: 15/892,747

(22) Filed: Feb. 9, 2018

Related U.S. Application Data
(60) Provisional application No. 62/457,354, filed on Feb. 10, 2017.

(51) Int. Cl. A61B 17/00; A61K 8/64; A61Q 19/00; A61K 8/98; A61K 8/9789; A61K 8/92; A61K 8/73; A61K 8/37; A61K 8/55; A46B 5/02; A46B 9/02

(52) U.S. Cl. CPC A61B 17/00; A61K 8/64; A61Q 19/001; A61K 8/987; A61K 8/9789; A61B 2017/320012; A61K 8/735; A61K 8/37; A61K 8/553; A46B 5/021; A46B 9/02; A61K 8/922

(57) ABSTRACT

An exfoliating tool to cleanse and exfoliate the lips and other areas of the face, and a lip balm to soften and plump lips.

### CROSS-REFERENCE TO RELATED APPLICATIONS

This application claims priority to and the benefit of U.S. Provisional Patent Application Ser. No. 62/457,354 filed Feb. 10, 2017, the disclosure of which is incorporated herein by reference in its entirety.

### FIELD

The aspects of the present disclosure embodiments generally relate to facial and skin care products, and in particular to an exfoliating tool for the lips and an exfoliating lip balm.

### BACKGROUND

Skin continuously generates new skin cells. The generation of new skin cells occurs in the dermis layer from which they are sent to the epidermis, the outer most skin layer. As the cells proceed to the epidermis they gradually die and become filled with keratin. These keratinized skin cells are important because they give our skin a protective quality, however, such dead cells are constantly sloughing off to make way for younger cells. This process of cell turnover slows down as we get older and cells start to build up unevenly on the skin's surface. Such build up can give older skin a dry and rough appearance.

In order to remove the buildup of dead skin cell, exfoliation can be used to remove such skin cells resulting in the younger skin cells being visible and the skin exhibiting a younger and more vibrant appearance. A person's lips are particularly susceptible to a dry and scaly appearance resulting from the accumulation of dead skin cells. Current lip treatments only soften the dead skin layer, but they do not remove this flaky and unsightly layer.

There is a need for an effective yet gentle exfoliant tool capable of removing dead skin cells accumulating on the lips as well as a lip balm to treat the lips, for example, post-exfoliation.

### SUMMARY

In one embodiment, the aspects of the present disclosure are directed to an exfoliant tool for cleansing and exfoliating a skin surface of an animal subject. The exfoliant tool includes a head portion, a handle member attached to the head portion and a plurality of bristles extending from a surface of the head portion and oriented generally perpendicular to the surface of the head portion.

In another embodiment, the aspects of the present disclosure are directed to a lip balm including a moisturizer and a modified matrikine-mimetic peptide to plump lips by hyaluronic acid synthesis.

In another embodiment, the aspects of the present disclosure are directed to a method of exfoliating a lip surface with an exfoliant tool. The exfoliant tool includes a head portion, a handle member attached to the head portion and a plurality of bristles extending from a surface of the head portion and oriented generally perpendicular to the surface of the head portion. The method includes dampening the lip surface with warm water, exfoliating the lip surface with the exfoliant tool, rinsing the lip surface with warm water and drying the lip surface.

### BRIEF DESCRIPTION OF THE DRAWINGS

These and other features, aspects, and advantages of the present disclosure will become better understood when the following detailed description is read with reference to the accompanying drawings in which like characters represent like parts throughout the drawings, wherein:

FIG. 1 is a top perspective view of one embodiment of a lip exfoliant tool;

FIG. 2 is a first side view of the lip exfoliant tool of FIG. 1;

FIG. 3 is a bottom view of the lip exfoliant tool of FIG. 1;

FIG. 4 is a top view of the lip exfoliant tool of FIG. 1; and

FIG. 5 is a second side view of the lip exfoliant tool of FIG. 1.

### DETAILED DESCRIPTION

As used herein, the term “exfoliation” refers to a detachment and shedding of superficial cells of an epithelium layer from a tissue surface, such as, for example, the skin surface of an animal subject, such as, for example, a human being.

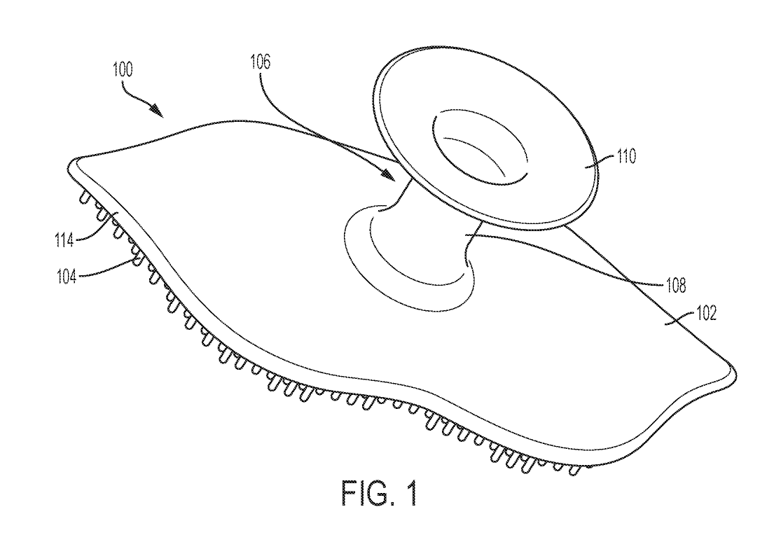

Referring to FIG. 1, the aspects of the disclosed embodiments are directed to an exfoliant tool 100 suitable for use on the lips. For the purposes of the disclosure herein, the tool 100 will be referred to as a “brush” or “sponge.” The brush 100 of the disclosed embodiments can be utilized to accomplish the process of lip cleansing and exfoliation of a human user.

As is illustrated in FIG. 1, the brush 100 generally includes a head portion 104 and a handle member 106. The head portion 102 includes upstanding members 104, generally referred to as “bristles” herein. The bristles 104 are attached to or integral to the head portion 102. The head portion 102 may be flexible or rigid.

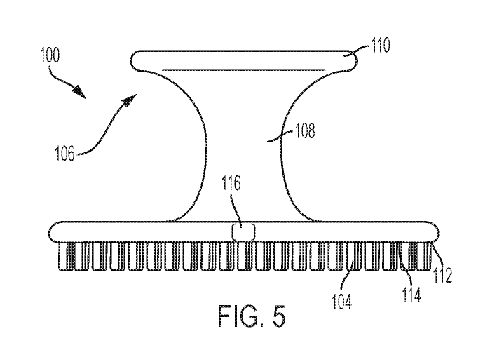

An exemplary ornamental shape of the tool 100, and in particular the head portion 102 is illustrated in FIGS. 1-5. In this example, the shape of the head portion 102 is in the general form or shape of a pair of lips. In alternate embodiments, the shape of the head portion 102 can be any suitable shape, such as, for example, square, rectangular, round, elliptical curved. Generally, the shape of the head portion 102 is to enable adequately placing the bristles 104 in physical contact with the facial surfaces, and in particular the areas of the lips.

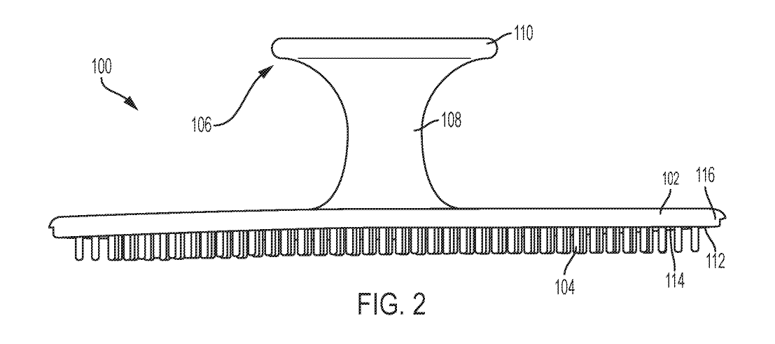

As is shown in FIGS. 2, 3 and 5, for example, the bristles 104 are arranged or oriented in a generally perpendicular manner relative to a surface 112 of the head portion 102. In alternate embodiments, the bristles 104 can be arranged at any suitable angle relative to the surface 112 of the head portion 102 so as to place the bristles 104 in a generally perpendicular angle relative to the lip surface being treated.

In one embodiment, the bristles 104 may be soft and flexible, but also possess sufficient stiffness or rigidity to promote and provide gentle exfoliation of a lip surface without scratching, cutting, gouging, or otherwise damaging the lips. The bristles 104 may comprise an appropriate polymer, such as, for example, silicone or a natural fiber material.

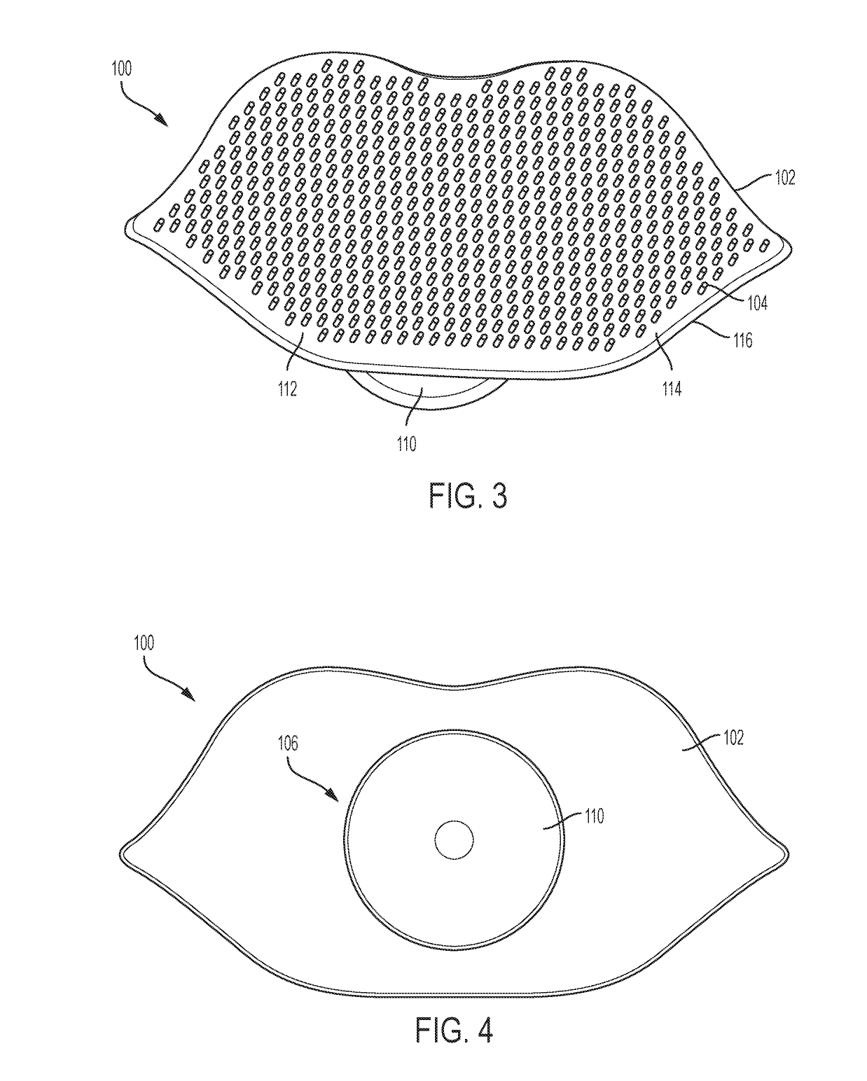

The bristles 104 can have a length that generally ranges from approximately 2 mm. to approximately 3 mm., preferably approximately 2.5 mm. and preferably the bristles are all of approximately the same length. As is illustrated in FIG. 3 the bristles 104 may be arranged in a plurality of rows and columns ranging in density from about 200 to 400 in number per square inch, preferably about 324 in number per square inch and preferably density of the bristles is substantially uniform over surface 112. For example, in FIG. 3 includes about 38 rows bristles across from left to right, with about 18 rows of bristles across from left to right in about 1 inch. In alternate embodiments, the bristles 104 can be arranged in any suitable pattern or patterns, other than including rows and columns.

In one embodiment, the head portion 102 includes a handle member 106. The handle member 106 is attached to or integral with the head portion 102. In one embodiment, a neck member 108 extends from the head portion 102 to a knob member 110. One end of the neck member 108 is attached to or integral with the head portion 102. The other end of the neck member 108 forms the knob 110. The knob 110 is generally used to hold or grip the tool 100. Although a knob is generally described herein, in alternate embodiments any suitable member can be used to grasp or hold the tool 100 using the handle member, other than including a knob. For example, the knob 110 can have a suitable shape and dimension so that a human hand, such as for example, a human user, can be used to manipulate the tool 100 to impart an exfoliating motion to the head portion 102 as it contacts the skin or lips. In one embodiment, the knob 110 can be configured to enable a mechanical appliance to hold the knob 110 and impart the exfoliating motion to the lip exfoliant brush to accomplish the process of lip exfoliation.

The tool 100 of the disclosed embodiments can be made from silicone and be anti-microbial. The surface 112 of the head portion 102 where the bristles 104 are located may include a circumferential margin space 114 in between a circumferential edge 116 of the head portion 102 and the bristles 104.

One embodiment for the process of lip exfoliation may include dampening the lip surface to be exfoliated with warm water, gently exfoliating the lip surface with an embodiment of the exfoliant brush 100, rinsing the lip surface with warm water and drying the lip surface, using, for example, a towel to, for example, pat the lips dry. The step of exfoliating the lip surface with the exfoliant brush 100 may include manipulating the bristles 104 of the exfoliant brush 100 along the surface of the lip by, for example, the user holding the handle member 106 of the exfoliant brush 100 and moving the brush in a linear, circular, elliptical or other motion or combination of motions so that the lip surface encounters the bristles 104 while the motion is applied thereto to effect exfoliation of the lips themselves. The motion may also include movement of the exfoliant brush 100, movement of the lips or a combination of both. An alternative embodiment for skin exfoliation may include a similar process to the one above, but used for treating a skin surface other than the lips.

Another embodiment may include applying a lip balm to the lip areas exfoliated subsequent to the exfoliation process or at other times when treatment of the lips is desired. The lip balm may have various cosmetic and therapeutic benefits that are imparted to the lip surface and subsequent tissue layers thereof treated therewith. The lip balm may include a moisturizer and an ingredient to plump the lips. The moisturizer may impart hydration to the lips and can include, for example, Cera Alba (beeswax), Butyrospermum Parkii (Shea Butter), Simmondsia chinensis (Jojoba) Seed Oil, caprylic/capric Triglyceride, Caryodendron orinocense Nut Oil or combinations thereof. The moisturizer may also impart other physical/cosmetic benefits and properties as well. One embodiment of the lip balm may contain shea butter, hyaluronic acid and Volulip™ to soften and plump lips and may be petroleum free. The lip balm may also include additional ingredients including vitamins, for example, such as Tocopheryl Acetate (which may also act as an antioxidant and antioxidants such as various plant extracts including, for example, Helianthus annuus (Sunflower) Seed Oil and Prunus dulcis (Almond) oil. These antioxidants may also impart other physical/cosmetic benefits and properties as well including for example, skin moisturizing and hydration. A therapeutic and/or cosmetically effective amount of the lip balm may be applied as needed, in single or multiple applications, directly to the lips from lip balm container or by applying the lip balm to an application device, such as the user's finger or a cotton swab, which, in turn is used to apply the lip balm to the lips.

Another embodiment of the lip balm may include the following ingredients: Butyrospermum Parkii (Shea Butter), hyaluronic acid, cetearyl ethylhexanoate, sorbitan isostearate, Portulaca pilosa extract, sucrose cocoate and the modified matrikine-mimetic peptide includes palmitoyl tripeptide-38.

Another embodiment of the lip balm may include the following ingredients: about 22 percent by weight (% by wt.) Butyrospermum Parkii (Shea Butter), about 0.1 percent by weight hyaluronic acid, about 0.1 percent by weight cetearyl ethylhexanoate, about 0.1 percent by weight sorbitan isostearate, about 0.1 percent by weight Portulaca pilosa extract, about 0.1 percent by weight sucrose cocoate and the modified matrikine-mimetic peptide includes about 0.1 percent by weight palmitoyl tripeptide-38.

Another embodiment of the lip balm may include the following ingredients: about 21.58 percent by weight Butyrospermum Parkii (Shea Butter), about 0.05 percent by weight hyaluronic acid, about 0.05 percent by weight cetearyl ethylhexanoate, about 0.05 percent by weight sorbitan isostearate, about 0.05 percent by weight Portulaca pilosa extract, about 0.05 percent by weight sucrose cocoate and the modified matrikine-mimetic peptide includes about 0.05 percent by weight palmitoyl tripeptide-38.

Another embodiment of the lip balm may include the following ingredients: bis-diglycerylpolyacyladipate-2, Cera Alba (beeswax), Butyrospermum Parkii (Shea Butter), Simmondsia chinensis (Jojoba) seed oil, Helianthus annuus (Sunflower) seed oil, caprylic/capric triglyceride, Prunus dulcis (Almond) oil, fragrance, lecithin, cetearyl ethylhexanoate, sorbitan isostearate, Portulaca pilosa extract, sucrose cocoate, palmitoyltripeptide-38, sodium hyaluronate, tocopheryl acetate and Caryodendron orinocense nut oil.

Another embodiment of the lip balm may include the following ingredients: about 29 percent by weight bis-diglycerylpolyacyladipate-2, about 23 percent by weight Cera Alba (beeswax), about 22 percent by weight Butyrospermum Parkii (Shea Butter), about 12 percent by weight Simmondsia chinensis (Jojoba) seed oil, about 11 percent by weight Helianthus annuus (Sunflower) seed oil, about 2 percent by weight caprylic/capric triglyceride, about 1 percent by weight Prunus dulcis (Almond) oil, about 0.5 percent by weight fragrance, about 0.5 percent by weight lecithin, about 0.1 percent by weight cetearyl ethylhexanoate, about 0.1 percent by weight sorbitan isostearate, about 0.1 percent by weight Portulaca pilosa extract, about 0.1 percent by weight sucrose cocoate, about 0.1 percent by weight palmitoyl tripeptide-38, about 0.1 percent by weight sodium hyaluronate, about 0.1 percent by weight tocopheryl acetate and about 0.1 percent by weight Caryodendron orinocense nut oil.

Another embodiment of the lip balm may include the following ingredients: about 29.56 percent by weight bis-diglycerylpolyacyladipate-2, about 22.89 percent by weight Cera Alba (beeswax), about 21.58 percent by weight Butyrospermum Parkii (Shea Butter), about 11.8 percent by weight Simmondsia chinensis (Jojoba) seed oil, about 10.66 percent by weight Helianthus annuus (Sunflower) seed oil, about 1.46 percent by weight caprylic/capric triglyceride, about 0.8 percent by weight Prunus dulcis (Almond) oil, about 0.5 percent by weight fragrance, about 0.37 percent by weight lecithin, about 0.05 percent by weight cetearyl ethylhexanoate, about 0.05 percent by weight sorbitan isostearate, about 0.05 percent by weight Portulaca pilosa extract, about 0.05 percent by weight sucrose cocoate, about 0.05 percent by weight palmitoyl tripeptide-38, about 0.05 percent by weight sodium hyaluronate, about 0.04 percent by weight tocopheryl acetate and about 0.04 percent by weight Caryodendron orinocense nut oil.

Volulip™ is an active substance manufactured by Sederma containing a plant extract and a natural modified matrikine-mimetic peptide to plump lips by hyaluronic acid synthesis. The ingredients include cetearyl ethylhexanoate, sorbitan isostearate, Portulaca pilosa extract, sucrose cocoate and palmitoyl tripeptide-38, the latter being the modified matrikine-mimetic peptide. It provides improvement of the internal and superficial lip texture with a visible impact on the lip shape. It also provides increase of the lip volume, hydration and softness as well as an anti-aging and anti-sagging effect.

Another embodiment is a kit that includes an embodiment of the exfoliant tool and an embodiment of the lip balm. Still another embodiment is a kit that includes an embodiment of the exfoliant tool, an embodiment of the lip balm and instructions for use.

### Lip Balm Example

| Trade Name | INCI Name | % by wt. | Function | Source |
|---|---|---|---|---|
| Softisan | Bis-Diglyceryl | 29.56 | Skin conditioning | Synthetic |
| 649 | Polyacyladipate-2 | | agent-emollient | |
| Beeswax | Cera Alba | 22.89 | Binder, Emulsion | Synthetic |
| | (beeswax) | | stabilizer, Viscosity | |
| | | | increasing agent- | |
| | | | nonaqueous | |
| Shea | Butyrospermum | 21.58 | Skin conditioning | Plant |
| Butter | Parkii (Shea | | agent-miscellaneous, | |
| | Butter) | | Skin conditioning | |
| | | | agent-occlusive, | |
| | | | Viscosity increasing | |
| | | | agent-nonaqueous, | |
| Jojoba Oil | Simmondsia | 11.8 | Hair conditioning | Plant |
| | Chinensis | | agent; Skin | |
| | (Jojoba) Seed | | conditioning agent- | |
| | Oil | | occlusive | |
| Sunflower | Helianthus | 10.66 | Skin conditioning | Plant |
| Oil | Annuus | | agent-miscellaneous, | |
| | (Sunflower) | | Sunscreen agent | |
| | Seed Oil | | | |
| Liponate | Caprylic/Capric | 1.46 | Skin conditioning | Plant, |
| GC | Triglyceride | | agent-occlusive | Synthetic |
| Sweet | Prunus dulcis | 0.8 | Skin conditioning | Plant |
| almond oil | (Almond) oil | | agent-emollient; Skin | |
| | | | Protectant | |
| Fragrance | Fragrance | 0.5 | Fragrance ingredient | |
| Lecithin | Lecithin | 0.37 | Skin conditioning | Animal, Plant |
| | | | agent-miscellaneous; | |
| | | | Surfactant- | |
| | | | emulsifying agent | |
| Vollulip | Cetearyl | 0.05 | Skin conditioning | Animal, Plant, |
| | Ethylhexanoate | | agent-emollient | Synthetic |
| Vollulip | Sorbitan | 0.05 | Surfactant- | Animal, Plant |
| | Isostearate | | emulsifying agent | |
| Vollulip | Portulaca Pilosa | 0.05 | Skin conditioning | Plant |
| | Extract | | agent-miscellaneous | |
| Vollulip | Sucrose | 0.05 | Skin conditioning | Plant, |
| | Cocoate | | agent-emollient, | Synthetic |
| | | | Surfactant- | |
| | | | emulsifying agent | |
| Vollulip | Palmitoyl | 0.05 | Skin conditioning | Synthetic |
| | Tripeptide-38 | | agent-miscellaneous | |
| Hyluronic | Sodium | 0.05 | Skin-conditioning | Animal, Plant, |
| acid | Hyaluronate | | agent-miscellaneous | Synthetic, |
| | | | | Bacteria, |
| | | | | Fungi or other |
| | | | | cell organism |
| Vitamin E | Tocopheryl | 0.04 | Antioxidant, Skin | Synthetic |
| Acetate | Acetate | | conditioning agent- | |
| | | | Miscellaneous | |
| Cacay Oil | Caryodendron | 0.04 | Skin conditioning | Plant |
| | orinocense Nut | | agent-emollient | |
| | Oil | | | |
| | Total | 100 | | |

This written description uses examples as part of the disclosure, including the best mode, and also to enable any person skilled in the art to practice the disclosed implementations, including making and using any devices or systems and performing any incorporated methods. The patentable scope is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. An exfoliant tool for cleansing and exfoliating a skin surface of an animal subject, comprising:
a head portion;
a handle member attached to the head portion; and
a plurality of bristles extending from a surface of the head portion and oriented generally perpendicular to the surface of the head portion.

2. The exfoliant tool according to claim 1, wherein a shape of the head portion is in a form of a pair of lips.

3. The exfoliant tool according to claim 1, wherein the head portion is flexible.

4. The exfoliant tool according to claim 1, wherein the head portion is rigid.

5. The exfoliant tool according to claim 1, wherein each bristle of the plurality of bristles is flexible with sufficient stiffness to promote and provide exfoliation.

6. The exfoliant tool according to claim 1, wherein each bristle of the plurality of bristles has a length ranging from about 2 mm. to about 3 mm.

7. The exfoliant tool according to claim 1, wherein the plurality of bristles are arranged in a plurality of rows and columns and have a density ranging from about 200 to 400 in number per square inch.

8. The exfoliant tool according to claim 1, wherein the handle member includes a neck member that extends from the head portion and a knob member that is connected to the neck member. the handle member having a suitable shape and dimension to be grasped by a human hand in order to manipulate the exfoliant tool.

9. A lip balm, comprising:
a moisturizer; and
a modified matrikine-mimetic peptide to plump lips by hyaluronic acid synthesis.

10. The lip balm according to claim 9, wherein the modified matrikine-mimetic peptide includes palmitoyl tripeptide-38.

11. The lip balm according to claim 9, wherein the moisturizer includes Cera Alba (beeswax), Butyrospermum Parkii (Shea Butter), Simmondsia chinensis (Jojoba) Seed Oil, caprylic/capric Triglyceride, Caryodendron orinocense Nut Oil or combinations thereof.

12. The lip balm according to claim 9, further including Butyrospermum Parkii (Shea Butter), hyaluronic acid, cetearyl ethylhexanoate, sorbitan isostearate, Portulaca pilosa extract, sucrose cocoate and the modified matrikine-mimetic peptide includes palmitoyl tripeptide-38.

13. The lip balm according to claim 9, including bis-diglycerylpolyacyladipate-2, Cera Alba (beeswax), Butyrospermum Parkii (Shea Butter), Simmondsia chinensis (Jojoba) seed oil, Helianthus annuus (Sunflower) seed oil, caprylic/capric triglyceride, Prunus dulcis (Almond) oil, fragrance, lecithin, cetearyl ethylhexanoate, sorbitan isostearate, Portulaca pilosa extract, sucrose cocoate, palmitoyltripeptide-38, sodium hyaluronate, tocopheryl acetate and Caryodendron orinocense nut oil.

14. The lip balm according to claim 9, including about 29 percent by weight bis-diglycerylpolyacyladipate-2, about 23 percent by weight Cera Alba (beeswax), about 22 percent by weight Butyrospermum Parkii (Shea Butter), about 12 percent by weight Simmondsia chinensis (Jojoba) seed oil, about 11 percent by weight Helianthus annuus (Sunflower) seed oil, about 2 percent by weight caprylic/capric triglyceride, about 1 percent by weight Prunus dulcis (Almond) oil, about 0.5 percent by weight fragrance, about 0.5 percent by weight lecithin, about 0.05 percent by weight cetearyl ethylhexanoate, about 0.05 percent by weight sorbitan isostearate, about 0.05 percent by weight Portulaca pilosa extract, about 0.05 percent by weight sucrose cocoate, about 0.05 percent by weight palmitoyltripeptide-38, about 0.05 percent by weight sodium hyaluronate, about 0.05 percent by weight tocopheryl acetate and about 0.05 percent by weight Caryodendron orinocense nut oil.

15. A method of exfoliating a lip surface with an exfoliant tool, wherein the exfoliant tool comprises:
a head portion;
a handle member attached to the head portion; and
a plurality of bristles extending from a surface of the head portion and oriented generally perpendicular to the surface of the head portion.
the method comprising:
dampening the lip surface with warm water;
exfoliating the lip surface with the exfoliant tool;
rinsing the lip surface with warm water; and
drying the lip surface.

16. The method according to claim 15, wherein the step of exfoliating the lip surface includes manipulating the bristles of the exfoliant tool along the lip surface by a user of the exfoliant tool, the user holding the handle member of the exfoliant tool and moving the exfoliant tool in a linear motion, circular motion, elliptical motion or a combination of motions so that the lip surface encounters the bristles while motion is applied to effect exfoliation of the lip surface.

17. The method according to claim 15, further including applying a lip balm to the lip surface subsequent to the drying step, the lip balm comprising:
a moisturizer; and
a modified matrikine-mimetic peptide to plump lips by hyaluronic acid synthesis.

18. The method according to claim 15, wherein each bristle of the plurality of bristles of the exfoliant tool is flexible with sufficient stiffness to promote and provide exfoliation.

19. The method according to claim 15, wherein the handle member of the exfoliant tool includes a neck member that extends from the head portion and a knob member that is connected to the neck member. the handle member having a suitable shape and dimension to be grasped by a human hand in order to manipulate the exfoliant tool.

20. The method according to claim 17, wherein the modified matrikine-mimetic peptide includes palmitoyl tripeptide-38.
